(19) United States
(12) Patent Application Publication
(10) Pub. No.: US 2008/0161688 A1
(43) Pub. Date: Jul. 3, 2008

(54) Portable Ultrasonic Diagnostic Imaging System with Docking Station

(75) Inventor: McKee Dunn Poland, Andover, MA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/911,119

(22) PCT Filed: Mar. 31, 2006
(86) PCT No.: PCT/IB06/50985

§ 371 Date: Oct. 10, 2007

Related U.S. Application Data
(60) Provisional application No. 60/672,625, filed on Apr. 18, 2005.

(51) Int. Cl. A61B 8/00

(52) U.S. Cl. 600/437

(57) ABSTRACT

An ultrasonic diagnostic imaging system is described which is operable in the docked mode or in the portable mode. In the docked mode a portable ultrasound system (60) is docked with a docking station (50). The portable ultrasound system (60) is controlled by hard key controls on a control panel of the docking station (50) and ultrasound images are displayed on a docking station display (28). In the portable mode the portable ultrasound system is operated separate from the docking station (50). Functions of a plurality of the hard keys of the control panel (44) are mapped to graphically displayed soft keys displayed on a flat panel display of the portable ultrasound system. The ultrasound system is controlled by clicking on these displayed soft keys or touching the displayed soft keys when the flat panel display is a touchscreen display.

This invention relates to portable ultrasonic diagnostic imaging systems and, in particular, to portable ultrasound systems operable with a cart-like docking station.

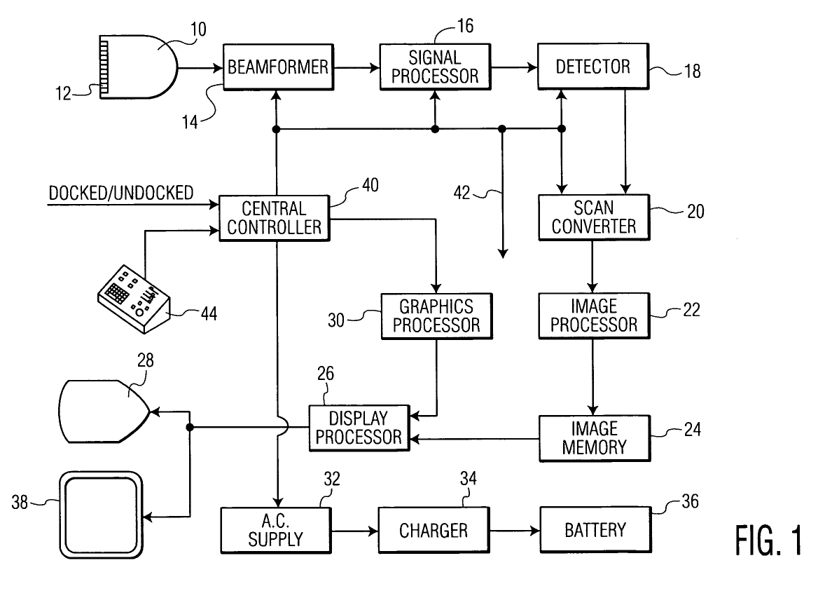

As semiconductor devices have become more miniaturized and capable of ever-increasing functionality, it has become possible to produce ever-smaller ultrasonic imaging devices. This reduction in size was initially made possible by the personal computer (PC), which provided significant processing power in a desktop unit. U.S. Pat. No. 6,063,030 (Vara et al.) shows one of the earliest efforts at using a PC as the core of a desktop ultrasound system, and illustrates how one of the limitations of a PC-based ultrasound system can be approached. A conventional cart-borne ultrasound system has a control panel and display, commonly referred to as the user interface, with a large number of hard and soft controls designed specifically for the operation of the ultrasound system. But when the same functionality is to be realized in a PC-like system, it is desirable to use the PC user interface as an ultrasound user interface, avoiding the additional cost and complexity of specialized hardware controls. In the '030 patent Vara et al. show in FIG. 1 the implementation of most of the ultrasound system controls as soft controls on the screen of the PC display screen 10. Vara et al. operate these soft controls with a mouse, keyboard key, or other pointing device to select and change the operation of their PC-like ultrasound system. In later years the graphical user interface concepts of Vara et al. were applied to the full range of PC-like devices. For example, Grunwald et al. have applied these user interface concepts to a PC, a laptop computer, and a personal digital assistant (PDA) in their US patent application pub. no. US 2004/0138569. Grunwald et al. obviate the need for a mechanical pointing device in many of their embodiments through the use of a display screen with touch-sensitive control areas, a touchscreen. By touching the visual display of a button or key on the screen the functionality of that button or key is invoked. European patent application pub. EP 1 239 396 A2 (Lifshitz et al.) envisions a medical imaging system where the touch-sensitive control areas of a touchscreen provide the complete control needed for the medical imaging system.

The ability to realize ultrasound functionality in a laptop computer or PDA device has led to the development of small, highly portable ultrasound imaging devices. A drawback of these small devices is that physicians more familiar with conventional cart-borne ultrasound systems often find the smaller devices uncomfortable to use. This shortcoming has been addressed by providing cart-like docking stations which provide some of the feel of using a cart-borne system. In U.S. Pat. No. 6,447,451 (Wing et al.), for instance, a fully integrated portable ultrasound system 10-14 can be mounted and operated on a docking stand 16-22. This approach still requires the physician to manipulate the small controls of the portable ultrasound system and use the small display screen of the portable system, however. This shortcoming is addressed in US patent application pub. no. US 2004/0150963 (Holmberg et al.), where a docking stand is provide with its own CRT or flat panel display monitor, larger user interface devices, and storage for peripheral devices. US patent application pub. no. US 2004/0179332 (Smith et al.) carry this concept a step further, providing a docking station 14 which substantially duplicates a full cart-borne system with a compartment in which a laptop-like portable system 12 can be inserted. The laptop-like portable system is fully operable as a portable ultrasound system with its own display screen 58 and user interface controls 30. When the portable system is mounted in the docking station 14 its display screen and user interface controls are inaccessible, at which time the operator uses the display screen 28 and full-size user interface 134 of the docking station to operate the system. Smith et al. recognize that the limited space and weight requirements of the portable system 12 mandate that only a subset of the full user control panel of the docking station be implemented on the portable system, and therefore choose to omit selected controls such as the TGC sliders from the portable ultrasound system user interface.

It is desirable to provide a portable ultrasound system which, when docked, can be operated with a complete set of full-size user interface controls, and when operated as a portable system, makes the same user interface controls available to the operator, thereby providing full ultrasound system functionality when docked or operated as a portable system.

In accordance with the principles of the present invention, a portable ultrasound system can be operated as a stand-alone, portable system or docked and operated in the manner of a cart-borne ultrasound system. When the portable system is docked the portable system senses this condition and allows the ultrasound functionality to be controlled by the user interface of the docking station. When the portable system is removed from the docking station and operated separately, the controls of the docking station which are not present as hard controls on the portable system are mapped to the graphical user interface and displayed and operated as soft controls. Thus, the full range of controls can be present in both the docked and portable modes of operation.

In the drawings:

FIG. 1 illustrates in block diagram form an ultrasonic diagnostic imaging system constructed in accordance with the principles of the present invention.

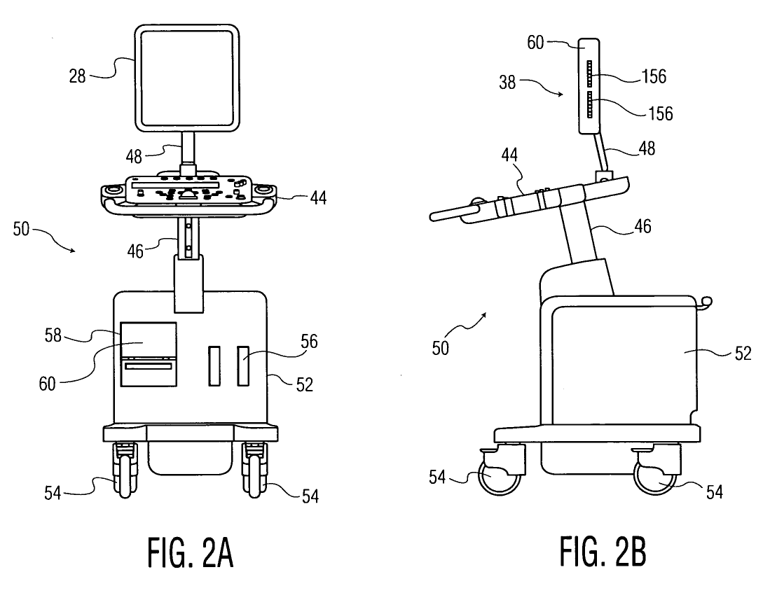

FIGS. 2a and 2b illustrate two embodiments of a portable ultrasound system docked in a cart-like docking station in accordance with the principles of the present invention.

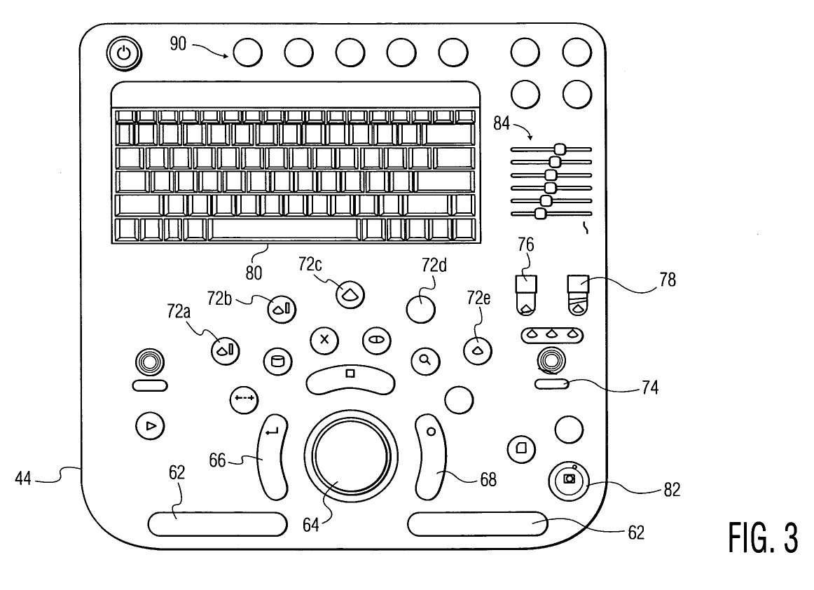

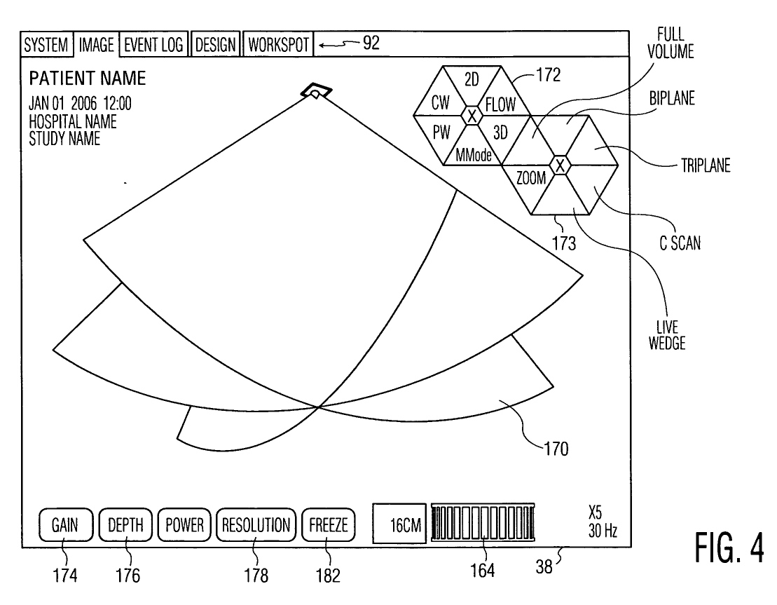

FIG. 3 illustrates a control panel user interface of a docking station of the present invention.

FIG. 4 illustrates a graphical user interface of a portable ultrasound system of the present invention.

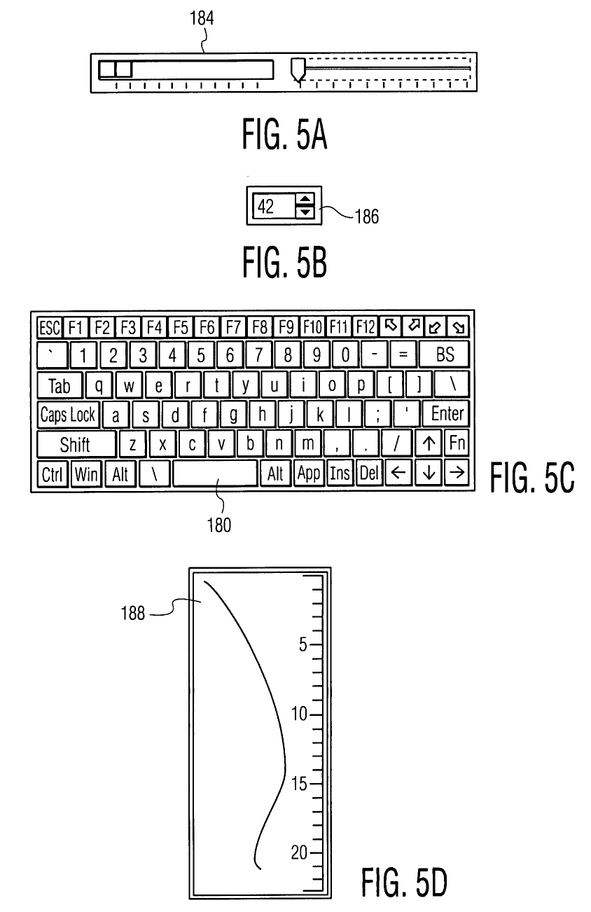

FIGS. 5a-5d illustrate hard controls of a docking station user interface which are realized as soft controls on a portable ultrasound system of the present invention.

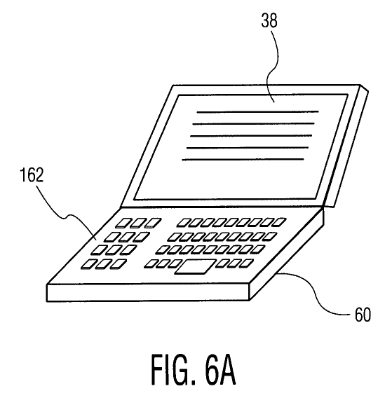

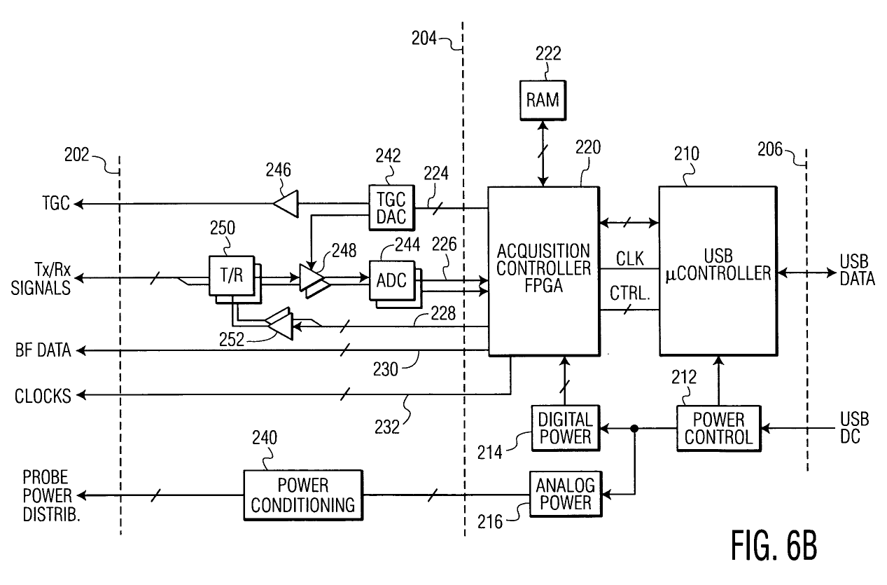

FIGS. 6a and 6b illustrate in block diagram form one embodiment of the acquisition subsystem of a portable ultrasound system of the present invention.

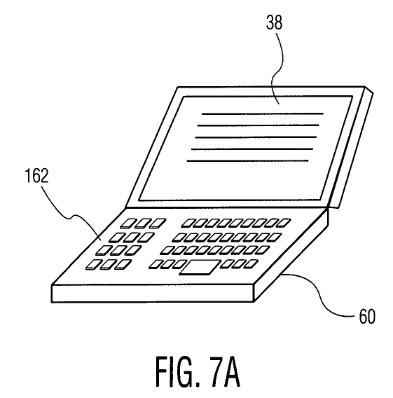

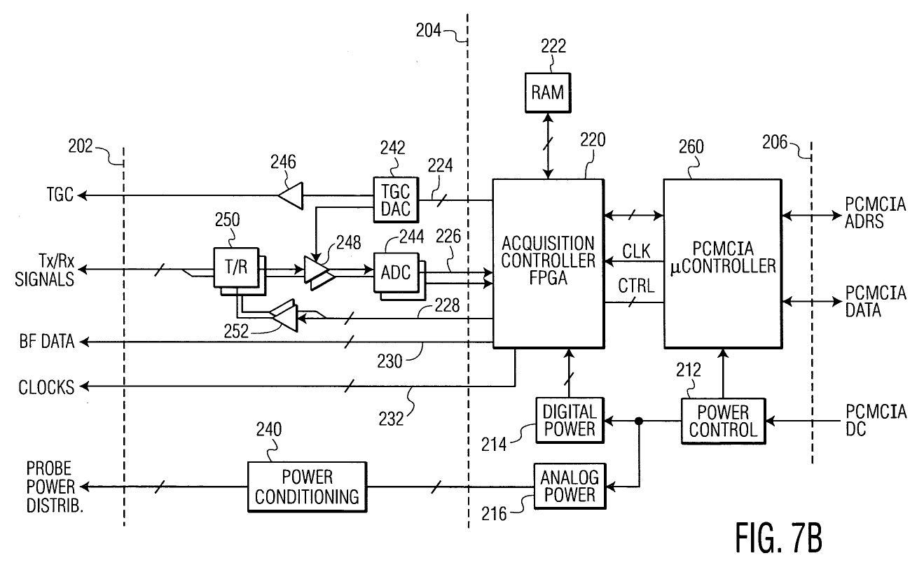

FIGS. 7a and 7b illustrate in block diagram form another embodiment of the acquisition subsystem of a portable ultrasound system of the present invention.

Referring first to FIG. 1, an ultrasonic diagnostic imaging system constructed in accordance with the principles of the present invention is shown in block diagram form. An ultrasound probe 10 transmits and receives ultrasound waves from the piezoelectric elements of an array of transducer elements 12. For imaging a planar region of the body a one-dimensional (1-D) array of elements may be used, and for imaging a volumetric region of the body a two-dimensional (2-D) array of elements may be used to steer and focus ultrasound beams over the image region. A transmit beamformer actuates elements of the array to transmit ultrasound waves into the subject. The signals produced in response to the reception of ultrasound waves are coupled to a receive beamformer 14. The beamformer delays and combines the signals from the individual transducer elements to form coherent beamformed echo signals. When the probe includes a 2-D array for 3D imaging, it may also include a microbeamformer which does partial beamforming in the probe by combining signals from a related group (“patch”) of transducer elements as described in U.S. Pat. No. 6,709,394. In that case the microbeamformed signals are coupled to the main beamformer 14 in the system which completes the beamforming process.

The beamformed echo signals are coupled to a signal processor 16 which processes the signals in accordance with the desired information. The signals may be filtered, for instance, and/or harmonic signals may be separated out for processing. The processed signals are coupled to a detector 18 which detects the information of interest. For B mode imaging amplitude detection is usually employed, whereas for spectral and color Doppler imaging the Doppler shift or frequency can be detected. The detected signals are coupled to a scan converter 20 where the signals are coordinated to the desired display format, generally in a Cartesian coordinate system. Common display formats used are sector, rectilinear, and parallelogram display formats. The scan converted signals are coupled to an image processor for further desired enhancement such as persistence processing. The scan converter may be bypassed for some image processing. For example the scan converter may be bypassed when 3D image data is volume rendered by the image processor by direct operation on a 3D data set. The resulting two dimensional or three dimensional image is stored temporarily in an image memory 24, from which it is coupled to a display processor 26. The display processor produces the necessary drive signals to display the image on a system image display 28 or the flat panel display 38 of the portable system. The display processor also overlays the ultrasound image with graphical information from a graphics processor 30 such as system configuration and operating information, patient identification data, and the time and date of the acquisition of the image.

A central controller 40 responds to user input from the user interface and coordinates the operation of the various parts of the ultrasound system, as indicted by the arrows drawn from the central controller to the beamformer 14, the signal processor 16, the detector 18, and the scan converter 20, and the arrow 42 indicating to the other parts of the system. The user control panel 44 is shown coupled to the central controller 40 by which the operator enters commands and settings for response by the central controller. The central controller 40 is also coupled to an a.c. power supply 32 to cause the a.c. supply to power a battery charger 34 which charges the battery 36 of the portable ultrasound system when the portable system is docked in the docking station.

In accordance with the principles of the present invention the central controller 40 is also responsive to a signal indicating whether the portable ultrasound system is docked or undocked, as indicated by the “Docked/Undocked” input to the central controller. This signal can be supplied by the operator pressing a Docked/Undocked button, a switch which changes state when the portable system is docked or undocked, or other suitable sensor of the docked/undocked condition. When the central controller is informed that the portable ultrasound system is docked in the docking station, the central controller responds to inputs from the user control panel 44, and causes the image to be displayed on the docking station display 28. The central controller also controls the graphics processor 30 during docking to omit the display of any softkey controls which duplicate the control functions of controls on the user control panel 44. The central controller may command the a.c. supply 32 and charger 34 to charge the battery 36 when the portable ultrasound system is docked, and/or power the docked portable system from a power supply on the docking station.

When the central controller is informed that the portable ultrasound system is undocked, these control characteristics are different. The controller now knows that user commands will not be received from the docking station control panel 44. The controller now causes some or all of the controls of the control panel 44 to be displayed when needed on the portable system display 38, as well as the ultrasound images produced by the ultrasound signal path. The a.c. supply 32 and the charger 34 are no longer controlled, as those subsystems are resident on the docking station. Probes will now be controlled through a probe connector on the portable system rather than through connectors on the docking station. The portable ultrasound system is now fully operable as a stand-alone ultrasound system.

It is thus seen that, in this embodiment, the partitioning of the components of FIG. 1 is as follows. The central controller 40, beamformer 14, signal processor 16, detector 18, scan converter 20, image processor 22, image memory 24, display processor 26, graphics processor 30, flat panel display 38, and battery 36 reside in the portable ultrasound system. The control panel 44, display 28, a.c. supply 32 and charger 34 reside on the docking station. In other embodiments the partitioning of these subsystems may be done in other ways as design objectives dictate.

FIGS. 2a and 2b illustrate two embodiments of a docking station 50 and portable ultrasound system constructed in accordance with the principles of the present invention. This docking station 50 greatly resembles a conventional cart-borne ultrasound system with a base unit 52 supporting the user control panel 44 on an adjustable support 46 which enables the control panel to be raised or lowered to accommodate the comfort of different users. The display 28 is mounted above the control panel 44, preferably on an adjustable support 48. An articulating adjustable support which serves this purpose is described in U.S. patent application Ser. No. 60/542,893 and international application no. PCT/IB2005/050405. The base unit 52 houses peripheral devices which the ultrasound system may use such as a printer, disk drive, and video recorder. The docking station 50 can be rolled to an exam room or patient bedside on wheels 54. The base unit also houses the a.c. power supply 32 and battery charger 34. The base unit may also have connections to connect the ultrasound system to a data network.

The base unit 52 has an enclosure 58 in the front into which a portable ultrasound system 60 can be located. When the portable ultrasound system 60 is inserted into enclosure 58 a connector on the portable system 60 engages a mating connector of the docking station. It is this engagement which, directly or indirectly, results in the “Docked” control signal being delivered to the central controller 40 of the portable system. The connector also provides the necessary connections to the control panel 44, the display 28, and the a.c. power supply 32, as well as the connection of the portable system battery 36 to the charger 34. This connector or another connector may also connect the portable system to one or more probe connectors 56 on the docking station. Alternatively, the probes may be connected to the portable system directly as they are in the portable mode, as by an opening on the side of the base unit 52 which permits the probe connector to directly engage probe connectors on the portable system 60.

In FIG. 2b the portable ultrasound system 60 is of the form of a notebook PC with a display screen 38 located on an outer surface of the portable ultrasound system. In this configuration the portable ultrasound system 60 is mounted in the position of the display 28 in FIG. 2a, and its display 38 is the display used when the portable system is docked on the docking station 50. Docking is done by mounting the portable ultrasound system to a connector on support 48. The portable ultrasound system thus is in communication with the docking station 50 by conductors passing through the support 48. In this view the portable ultrasound system probe connectors 156 can be seen on the side of the portable system 60. Probes can be connected to these connectors 156 or to connectors on the base unit 52 of the docking station if present.

FIG. 3 is a top plan view of a docking station control panel 44. The control panel 44 is seen to contain a number of buttons, switches and hard controls by which an operator controls the docked ultrasound system. In the front of the control panel 44 are two openings 62 which form handles at the front of the control panel. These handles are gripped when changing the height of the control panel by means of adjustable support 46. In the front center of the control panel is a trackball 64 which is used as a pointing device in conjunction with cursors and menus on the image display. To the left of the trackball is an “Enter” or “Return” key 66 which functions in the manner of the Enter key on a computer keyboard, and to the right of the trackball is a “Select” key 68 by which a user can select a menu item indicated by use of the trackball. In the lower right corner of the control panel is a “Freeze” button 82 which is used to capture or “freeze” a particular live image on the image display.

Above the trackball is an array of mode keys 72a-72e which are used to select a particular imaging mode of operation. These include the color Doppler mode selected by key 72a, the 2D (grayscale) mode selected by key 72c, the M-mode selected by key 72d, and so forth. On the right side of the control panel is the “Gain” button 74 by which the user can increase or decrease the gain of ultrasound signals used to produce the image. Increasing the gain may improve the image returned from greater tissue depths, for instance. Above the Gain button is a “Depth” button 76 that can be used to increase or decrease the depth of a displayed image. Next to the Depth button is a “Resolution” button 78 by which a user can enhance or increase the resolution of the ultrasound image. Above these controls is a full keyboard 80 which may comprise either mechanical or membrane keys. To the right of the keyboard 80 is a set of mechanical slide potentiometers which are used to set the TGC gain characteristic applied to the received echo signals.

In accordance with the principles of the present invention, when the docked ultrasound system 60 is undocked from the docking station and used as a stand-alone, portable ultrasound system, a number of the physical controls on the docking station control panel 44 are implemented as “soft” controls on the flat panel display screen 38 of the portable system. These soft controls may be actuated by a pointing device used to click on the soft controls on the display screen 38. The pointing device may be a physical device located on the control panel 162 of the portable system or located on the probe connected to the portable system where the sonographer can manipulate it with a finger while holding the probe. Alternatively, the flat panel display 38 may be a touchscreen display, enabling the user to select or manipulate the soft controls simply by touching their images on the display screen with a finger or implement in place of a mouse, trackball, or other electronic pointing device. On the lower left of the display screen 38 in FIG. 4 is a Gain softkey 174 which may be used to increase the signal gain, the same function performed by Gain button 74 on control panel 44. A Depth softkey 176 on the display screen enables the image depth to be changed, the same function as Depth button 76 on the docking station control panel. The image resolution may be changed with a Resolution softkey 178 which performs the function of control panel button 78, and a live image may be frozen by clicking or touching the Freeze softkey 182. To the right of these softkeys is a visual rotary or thumbwheel control 164. This softkey control may be dragged to the left or the right to rotate the planes of the tri-plane ultrasound image 170 about their common axis on the display screen. When the control panel 44 is used to control the display, the planes of the tri-plane image can be rotated by manipulation of the trackball 64 to rotate the planes at the back of the image to the front, for instance. When the display screen is a touchscreen, the tri-planes may be rotated simply by touching the screen and moving a finger from side to side in front of the tri-plane image. The thumbwheel control 164, like many others, is context-dependent, operating a function needed for the current image display.

In the upper right corner of the display screen 38 is a visual rendition of the mode selection keys 72n of the control panel 44. In this embodiment the mode selection keys are implemented as a softkey pie menu 172. A particular section of the pie may be selected by clicking or touching to select the indicated mode of operation. When a mode has a number of sub-modes, such as three dimensional (3D) imaging, selection of the mode opens up a submenu of more detailed selections. In this illustration the user has selected the 3D mode by means of pie menu 172 and, within the 3D mode and using submenu 173, has further selected the tri-plane submode. In the tri-plane submode, three planes through a volume are shown in perspective at the same time as illustrated by tri-plane display 170. The softkey rotary or thumbwheel control 164 or touching a touchscreen can then be used to rotate the planes about their common apex at the top of the display, causing image planes obscured at the back of the display to rotate to the front. Different softkeys can be displayed on the display screen 38 depending upon the image mode currently selected. The softkeys can be produced as static areas on a particular display or can be pulled down or pop up as menus when needed or called for.

FIGS. 5a-5d illustrate other forms that may be used for softkey controls on the portable ultrasound system display screen. FIG. 5a illustrates two softkey forms that may be used for the mechanical TGC sliders 84 of the control panel. In the form on the left the slider is a rectangle with a line down the center and in the form on the right the slider is a box which is drawn to a point on the bottom. The video slider can be dragged back and forth across the horizontal range of the slide control, just as the mechanical sliders on the control panel. FIG. 5b illustrates a softkey control which can be used when numerical indicators or accuracy are desired. This control is a spin-box 186 which shows a number which can be incremented or decremented by touching or clicking on the arrows to the right of the number. Such a control may be used for setting the depth of a displayed image, for example. FIG. 5c illustrates a full video QWERTY keyboard 180 which may be displayed and touched or clicked on to compose a text message or identifier or patient ID, for instance. Finally, FIG. 5d illustrates a profile curve 188 which may be touched or clicked on to drag portions of the curve to the left or right to change the characteristic of a corresponding TGC function for example. The depth scale to the right of the curve may also be dragged up or down in this example to extend or reduce the image depth. With this softkey, two functions, the TGC characteristic and the image depth, may be changed by means of one softkey display.

Table I illustrates the embodiment of a number of ultrasound control functions as hard controls on a docking station control panel, and as soft controls on an undocked (detached) portable ultrasound system. The ultrasound control functions are listed in the first column of the Table. Their implementation as hard controls is described in the center column, where “hard button” refers to a physical button on a control panel and “soft button” refers to one of the numbered buttons 90 at the top of the control panel (see FIG. 3) each of which is aligned with an area of the display screen immediately above the button. By displaying a programmable function label in a display screen area the function of the button can be made programmable for different functions during

TABLE I

| | Cart-Based Hard | Detached System |
|---|---|---|
| Control | Controls | Touch Graphic |
| TGC | Slide Pots | Profile Track Bar |
| Depth | Rocker Switch | Thumbwheel, readout |
| Focus | Rocker Switch | Spin Box, readout |
| Gain | Rotary Knob | Thumbwheel, readout |
| Freeze | Hard Button | Button |
| Scroll Image | Rotary Knob | Slider |
| Power | Rotary Knob | Thumbwheel, readout |
| Greyscale Mode | Hard Button | Pie Menu |
| Greyscale Submodes | Soft Button | Cascaded Pie Menu |
| Flow Mode | Hard Button | Pie Menu |
| Color Power Angio | Hard Button | Cascaded Pie Menu |
| Flow Submodes | Soft Button | Cascaded Pie Menu |
| PW Mode | Hard Button | Pie Menu |
| CW Mode | Hard Button | Pie Menu |
| 2D Mode | Hard Button | Pie Menu |
| 3D Mode | Hard Button | Pie Menu |
| 3D Submodes | Soft Button | Cascaded Pie Menu |
| ROI Movement | Hard Button + Trackball | Touch Drag |
| Acquire | Hard Button | Button |
| Caliper | Hard Button + Trackball | Button, Touch Points |
| Patient Entry | Hard Button | Tab Page |
| Setup | Hard Button | Tab Page |
| Probe Select | Soft Button | Tab Page, Button |
| Preset | Hard Button | Tab Page |
| Text Entry | QWERTY Keyboard | Touch Keyboard |

different modes of ultrasound system operation. The third column of Table I shows the control functions when implemented visually on the portable ultrasound system display. “Tab Page” refers to a visual control 92 located at the edge of the display screen (see FIG. 4) which may be touched or clicked to view a different display page. It is thus seen that a variety of familiar ultrasound system controls can be mapped from mechanical controls to visual controls for a portable system.

In one embodiment of the present invention the ultrasound probe comprises a matrix array probe as described in US Pat. Nos. 6,375,617 (Fraser et al.) and 5,997,479 (Savord et al.) The matrix array probe contains not only a transducer array but also microbeamformer circuitry which performs at least some of the beamforming of the signals received by the probe. A matrix array probe can also make efficient and compact use of a two-dimensional array transducer which can perform three dimensional imaging, either images of a volumetric region or of several planes occupying a volumetric region. When some of the beamforming is performed in the probe, a reduced processing burden is imposed on the ultrasound system to which the matrix probe is connected and operates.

FIG. 6a illustrates an embodiment of the portable ultrasound system 60 in which the portable system utilizes the conventional packaging of a laptop PC. By taking advantage of the processing power and existing packaging of a laptop PC, no specialized packaging components are needed, reducing the cost of the portable system. Much of the signal processing and all of the display processing and user interface control can be performed using the microprocessor(s) of the portable PC unit. In addition, connectors for interfacing laptop PCs to docking stations are well developed and commercially available, reducing that cost of system development. When realized in a laptop PC package use can be made of the conventional keyboard and controls 162 of the laptop PC including the touchpad or joystick pointing device commonly integrated into the laptop keyboard. The portable ultrasound system display 38 is provided by the conventional flat panel display 38 of the laptop PC, which is preferably modified to be at least partly or wholly a touchscreen display.

Another advantage of laptop or notebook PC packaging for the portable ultrasound system is the convenience of interfacing to the matrix array probe. FIG. 6b illustrates a first such interface in block diagram form. This probe interface is bounded by a vertical dashed line 202 on the left and a vertical dashed line 206 on the right. To the left of dashed line 202 is the matrix array probe, connected to the signal lines indicated by the arrows. To the right of dashed line 206 is the laptop or notebook PC system. In the embodiment of FIG. 6b the interface is connected to the standard lines of a USB connection, including the USB data lines and the USB DC (power) line shown to the right of dashed line 206. Thus, the ultrasound probe in this embodiment is interfaced to the portable PC by a standard USB interface compatible with an interface protocol already present on the portable PC, reducing the cost and complexity of the interface to the PC.

The probe-PC interface can be divided into two regions of data circuitry. The region between dashed lines 204-206 is a region of digital circuitry which may, if desired, be fabricated as a digital circuitry module. The region between dashed lines 202-204 may be viewed as a region of analog circuitry which may, if desired, be fabricated as an analog circuitry module. Alternately, both modules may be fabricated on a common printed circuit board. Such a board or boards can conveniently be located in a standard laptop PC compartment such as the extra battery or disk drive bay. Thus, the interface can be realized as modules which are located inside the case of the laptop PC rather than as a separate module box that is used between the probe and the portable PC.

The USB DC lines are coupled to power control circuitry 212 which distributes DC power to digital power circuitry 214 and analog power circuitry 216. The digital power circuitry 214 distributes power to the digital components of the digital module including in this embodiment a USB microcontroller 210 and an acquisition controller FPGA 220 and its accessory components such as RAM 222. The USB microcontroller 210 exchanges USB data with the portable PC over the USB data line and with the FPGA 220 over data, clock and control lines. The USB microcontroller is the means by which the FPGA and the portable PC communicate through a USB port. The acquisition controller FPGA (field programmable gate array) is a programmable hardware device that performs most or all of the ultrasound acquisition functions of the portable ultrasound system, such as transmit and receive beamforming, filtering, demodulation, harmonic separation and, if desired and given sufficient FPGA circuitry, amplitude and/or Doppler detection.

In the analog module the analog power circuitry 216 of the digital module is coupled to power conditioning circuitry 240 which distributes power to the components of the analog module and is also connected to provide power to the power distribution circuitry of the probe. The FPGA 220 provides beamformer data and clock signals for the microbeamformer of the matrix array probe on lines 230 and 232. In this embodiment these lines pass through the analog module for connection to the probe. Bipolar drive signals for the transducer elements of the probe are provided by the FPGA 220 on lines 228, amplified by amplifiers 252, and coupled to the probe by transmit/receive switches 250. Ultrasound signals received by the transducer elements of the probe are microbeamformed and amplified, then coupled through the transmit/receive switches 250 to TGC amplification stages 248. The TGC amplified signals are digitized by analog to digital converters (ADCs) 244 and coupled digitally to the FPGA over lines 226. TGC control is also effected by a TGC signal on lines 224 which is converted to an analog signal by TGC DAC 242, then distributed to TGC amplification stages 248 and to gain control circuitry in the probe by amplifier 246. A portion of the TGC control may also be performed digitally in the FPGA 220.

In a typical configuration the ultrasound signals received by dozens or hundreds of transducer elements in the probe are initially microbeamformed and combined down to a lesser number of ultrasound signal channels, such as sixteen or thirty-two channels. The final beamforming of these sixteen or thirty-two channels may be performed by the FPGA 220 when programmed for configuration as a sixteen-channel or thirty-two-channel receive beamformer. The final beamformed line signals, which may also undergo other signal processing in the FPGA as described above, are coupled to the portable PC over the USB interface for image processing and display on the display 38 of the portable ultrasound system. The portable ultrasound system is controlled by a user interface such as that illustrated in FIG. 4. When the portable ultrasound system 60 is docked in the docking station 50, the probe may be connected to the analog module by a multiplexer between probe connectors 56 on the docking station (when present) and the analog module by way of the docking connector between the docking station and the portable ultrasound system. When docked the ultrasound system is controlled by the control panel 44 with controls coupled to the docking connector and the ultrasound images are displayed on the docking station display 28 (FIG. 2a) or display 38 (FIG. 2b).

FIG. 7a again illustrates a portable ultrasound system 60 packaged as a portable PC. In this embodiment the digital communication between the acquisition system and the portable PC is by means of a parallel data interface rather than a serial data interface. This embodiment is configured with a PCMCIA interface between the FPGA 220 and the portable PC as shown in FIG. 7b. Most portable PCs have a connector slot for PCMCIA cards inside the case of the PC. This means that the digital module between dashed lines 204 and 206 can be fabricated as a PCMCIA card which is located in such a slot and communicates directly with the portable PC by way of its PCMCIA interface. Thus, no specialized parallel digital interface needs to be developed to communicate with the portable PC. The analog module can be located in a similar slot or in an accessory bay of the portable PC.

The PCMCIA interface includes a PCMCIA microcontroller 260 which is connected to PCMCIA address and data lines of the portable PC. The DC conductors of the PCMCIA interface are coupled to provide DC power to the power control circuitry 212. The FPGA 220 can thus communicate through the PCMCIA interface to receive programs and data from the portable PC and to forward acquired ultrasound data to the portable PC for display. The use of native PC interfaces of a laptop or notebook PC enables the production of an inexpensive and conveniently packaged portable ultrasound system 60.

What is claimed is:

1. An ultrasonic diagnostic imaging system including a portable ultrasound system which is operable in a docked mode with the portable ultrasound system connected to a docking station and a portable mode in which the portable ultrasound system is operable separate from the docking station comprising:
a hard key control panel connected to the docking station and including a plurality of hard key mechanical controls which act to control the ultrasound system when the portable ultrasound system is operated in the docked mode; and
a portable system display panel, connected to the portable ultrasound system and operable with the portable ultrasound system when the portable ultrasound system is operated in the portable mode, the portable system display panel displaying a plurality of softkey controls which are selectable by a user to control the portable ultrasound system when operated in the portable mode, the softkey controls performing the functions of corresponding hard key mechanical controls of the hard key control panel when the portable ultrasound system is operated in the portable mode.

2. The ultrasonic diagnostic imaging system of claim 1, wherein the display panel is inoperable when the portable ultrasound system is operated in the docked mode.

3. The ultrasonic diagnostic imaging system of claim 1, wherein the portable ultrasound system further includes a system controller which is responsive to the hard key mechanical controls of the hard key control panel to control the ultrasound system when the ultrasound system is operated in the docked mode, and is responsive to the selection and/or manipulation of the soft key controls on the portable system display panel to control the ultrasound system when the ultrasound system is operated in the portable mode.

4. The ultrasonic diagnostic imaging system of claim 1, wherein the portable ultrasound system further includes a pointing device which is operable to select and or manipulate a soft key control on the portable system display panel.

5. The ultrasonic diagnostic imaging system of claim 1, wherein the portable system display panel comprises a touchscreen display.

6. The ultrasonic diagnostic imaging system of claim 1, wherein the hard key control panel includes an alphanumeric keyboard and a plurality of mechanical controls having ultrasound-specific functionality; and
wherein the portable ultrasound system includes an alphanumeric keyboard and a plurality of soft key controls displayed on the portable system display panel which replicate the ultrasound-specific functionality of a plurality of the mechanical controls of the hard key control panel.

7. The ultrasonic diagnostic imaging system of claim 6, wherein the alphanumeric keyboard of the hard key control panel comprises a mechanical key alphanumeric keyboard.

8. The ultrasonic diagnostic imaging system of claim 7, wherein the alphanumeric keyboard of the portable ultrasound system comprises a mechanical key alphanumeric keyboard.

9. The ultrasonic diagnostic imaging system of claim 6, wherein all of the controls having ultrasound-specific functionality in the portable mode are implemented as soft key controls on the portable system display panel.

10. The ultrasonic diagnostic imaging system of claim 1, wherein the hard key control panel includes an alphanumeric keyboard and a plurality of mechanical controls having ultrasound-specific functionality; and
wherein the portable ultrasound system includes an alphanumeric keyboard and a plurality of soft key controls displayed on the portable system display panel which replicate an alphanumeric keyboard and the ultrasound-specific functionality of a plurality of the mechanical controls of the hard key control panel.

11. The ultrasonic diagnostic imaging system of claim 10, wherein the alphanumeric keyboard of the hard key control panel comprises a mechanical key alphanumeric keyboard.

12. The ultrasonic diagnostic imaging system of claim 11, wherein the alphanumeric keyboard of the portable ultrasound system comprises a mechanical key alphanumeric keyboard.

13. The ultrasonic diagnostic imaging system of claim 1, wherein the portable ultrasound system further includes a system controller which is responsive to the hard key mechanical controls of the hard key control panel to control the ultrasound system when the ultrasound system is operated in the docked mode, and is responsive to the selection and/or manipulation of the soft key controls on the portable system display panel to control the ultrasound system when the ultrasound system is operated in the portable mode; and
a graphics display subsystem which is responsive to the system controller and coupled to the portable system display panel for displaying soft key controls on the portable system display panel when the ultrasound system is operated in the portable mode.

14. The ultrasonic diagnostic imaging system of claim 1, wherein the docking station further includes a docking station display which acts to display ultrasound images in the docked mode,
wherein ultrasound images are displayed on the portable system display panel when the ultrasound system is operated in the portable mode.

15. A method for selectively operating a portable ultrasound system in a docked mode or a portable mode, the portable ultrasound system having a flat panel display, comprising:
connecting the portable ultrasound system to a docking station;
operating the ultrasound system by means of a docking station control panel which includes a plurality of hard key controls which control predetermined functionality of the ultrasound system when the system is operated in the docked mode;
disconnecting the portable ultrasound system from the docking station; and
operating the ultrasound system by means of a plurality of soft key controls displayed on the flat panel display when the system is operated in the portable mode, the soft key controls being mapped to control the same predetermined functionality as the corresponding hard key controls of the docking station control panel.

16. The method of claim 15, wherein the docking station further includes a display; and further comprising:
displaying ultrasound images on the docking station display when the system is operated in the docked mode; and
displaying ultrasound images on the flat panel display when the system is operated in the portable mode.
